(19) United States
(12) Patent Application Publication
(10) Pub. No.: US 2016/0198682 A1
(43) Pub. Date: Jul. 14, 2016

(54) Animal training apparatus and method

(71) Applicant: K-9 Search On Site, LLP, Oak Ridge, TN (US)

(72) Inventors: F. Shayne Brasfield, Oak Ridge, TN (US); Amy Brasfield, Oak Ridge, TN (US)

(21) Appl. No.: 14/757,596

(22) Filed: Dec. 23, 2015

Related U.S. Application Data
(60) Provisional application No. 62/095,856, filed on Dec. 23, 2014.

(51) Int. Cl. A01K 15/02; A61M 5/172; A61M 5/142

(52) U.S. Cl. CPC A01K 15/02; A01K 15/021; A61M 5/14244; A61M 5/172; A61M 2250/00; A61M 2205/18

(57) ABSTRACT

An injection apparatus and associated method for rapidly training an animal to associate a behavior conducted by the animal with rapid chemical feedback to the animal's bloodstream.

### PRIORITY

This application claims priority to U.S. provisional application Ser. No. 62/095,856 entitled “Animal Training Apparatus and Method” which was filed on Dec. 23, 2014, the entirety of which is incorporated herein by reference.

### FIELD

This disclosure relates to the field of training animals. More particularly, this disclosure relates to a method and apparatus for training an animal such as a canine to quickly learn to repeat desired behaviors under specific circumstances.

### BRIEF DESCRIPTION OF THE DRAWINGS

Further features, aspects, and advantages of the present disclosure will become better understood by reference to the following detailed description, appended claims, and accompanying figures, wherein elements are not to scale so as to more clearly show the details, wherein like reference numbers indicate like elements throughout the several views, and wherein:

FIG. 1 shows a somewhat schematic perspective view of a first example of an injection apparatus;

FIG. 2 shows a somewhat schematic perspective view of a second example of an injection apparatus;

FIG. 3 shows an injection apparatus including a torso pack; and

FIG. 4 shows an injection apparatus including a collar.

### DETAILED DESCRIPTION

Various terms used herein are intended to have particular meanings. Some of these terms are defined below for the purpose of clarity. The definitions given below are meant to cover all forms of the words being defined (e.g., singular, plural, present tense, past tense). If the definition of any term below diverges from the commonly understood and/or dictionary definition of such term, the definitions below control.

Air: broadly defined to include a scientific definition of “air” and/or other pure gases and gas mixtures and all solid, liquid, and gaseous substances entrained therein.

Fluid inducer: an apparatus (e.g., a pump, or other similar device, with or without moving mechanical parts) used to cause fluid to move relative to a conduit by pushing or pulling such fluid.

Animate Odor Detector: An animal that has been trained or otherwise has learned a specific behavioral response (or “trained signal”) when such animal detects a specific target odor. Animate odor detectors can include, for example, canines, mice, monkeys, and other animals capable of detecting a target odor(s) at very low concentrations and exhibiting learned or trained behavior based on the detection of such target odors(s).

Conduit: an apparatus configured to direct or otherwise channel gas and/or fluid from a first location to a second location.

Engagement Apparatus: an apparatus to be acted upon (i.e., “engaged”) directly or indirectly by an animate odor detector.

Fluid: broadly defined to include pure gases, gas mixtures, pure liquids, and liquid mixtures (including solids, liquids, and/or gases in any form entrained therein including, for example, mists and powders).

Gas: broadly defined to include pure gases and gas mixtures (including solids, liquids, and/or gases in any form entrained therein including, for example, mists and powders).

Target Odor: an odor of interest that may indicate the presence of a contraband substance such as, for example, illegal narcotics, explosives, chemical weapons, biological weapons, or anything deemed a potential threat to an area being secured.

Trained signal: a specific trained or learned behavioral response given by an animate odor detector in response to the animate odor detector detecting a target odor.

Triggering event: a situation in which an animate odor detector gives a trained signal, indicating that a target odor has been detected.

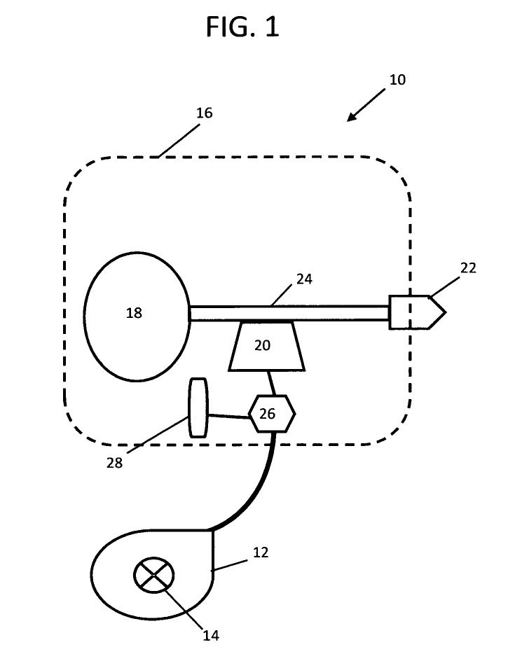

FIG. 1 shows an embodiment of an injection training apparatus 10 for rapidly training an animal to associate a behavior conducted by the animal with rapid chemical feedback to the animal's bloodstream. For example, the apparatus may be used to train an animal to identify a specific target odor and to rapidly demonstrate a trained signal after detection of the specific target odor. The behavior conducted by the animal that triggers feedback to the animal may be active or passive behavior depending on how the animal is being trained, and the feedback may be positive reinforcement or negative reinforcement behavior training,

The injection training apparatus 10 includes a triggering device 12 to trigger flow of a fluid through the injection apparatus. The triggering apparatus 12 includes an actuator 14 for initiating a fluid flow event wherein fluid is caused to enter the bloodstream of an animate odor detector.

A mounting platform 16 is also included which is mounted to an animal, preferably near a site wherein fluid is sent to interface with the bloodstream of the animal. The mounting platform 16 further includes a fluid storage chamber 18, a fluid inducer 20, an outlet 22, and a conduit 24 connected to the fluid storage chamber 18, the fluid inducer 20, and the outlet 22. The outlet 22 is configured for fluid to flow through it to enter the bloodstream of an animal on which the mountain platform 16 is mounted.

In one example the mounting platform 16 includes a simple controller (SISO) 26 connected to the actuator 14 and a first power source 28, preferably in the form of a removable or rechargeable battery. The actuator 14 is preferably a button provided on a leash wherein a wire runs from the button along the leash to the controller 26.

In one example, the outlet 22 includes an IV catheter and fluid is transferred directly into a vein or artery of the animal from the outlet 22. In another example, the outlet 22 includes a nasal discharge port were fluid is discharged into a nostril of an animal and quickly enters the bloodstream of the animal. In another embodiment, the outlet 22 goes into a muscle of the animal (e.g., the heart). In yet another embodiment, the outlet is located in the anal cavity of the animal for fluid to cross into the bloodstream through the animal's colon.

Fluids used for the purposes of training can range from stimulants to doping agents and include anything that would cause a quick chemical and biological reaction. Specific examples include cocaine, nicotine, sucrose and this list is not intended to be limiting as to what can be used to accomplish the goals taught or otherwise disclosed herein. Feedback to the animal may come in the form of a reward or a punishment, depending on the behavior displayed by the animal or the training regimen being used.

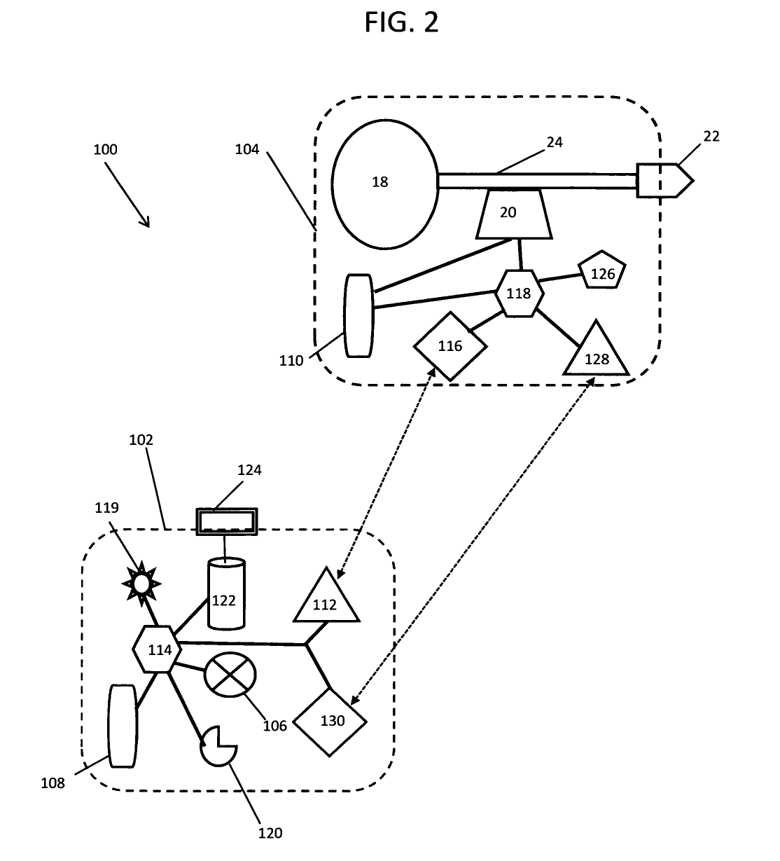

FIG. 2 shows a schematic view of an embodiment of an injection training apparatus 100 that includes a triggering device 102 and a mounting platform 104. The triggering device 102 includes an actuator 106 as described in more detail below as well as a first power source 108. The mounting platform 104 includes the fluid storage chamber 18; the fluid inducer 20; the outlet 22; and the conduit 24 as well as a second power source 110. Although the first power source 108 and the second power source 110 are not shown as being connected to all powered parts of the injection training apparatus 100, it is understood that those features of the apparatus 100 are powered by these power sources through the controllers 114 and 118, respectively. Alternatively, the various powered parts of the injection training apparatus 100 can be connected directly to the first power source 108 or the second power source 110.

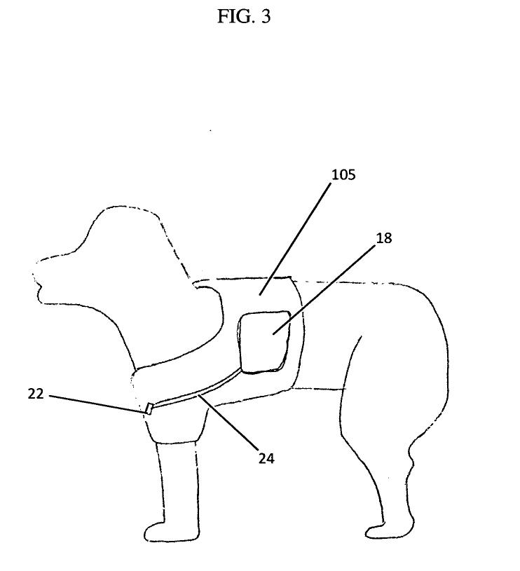

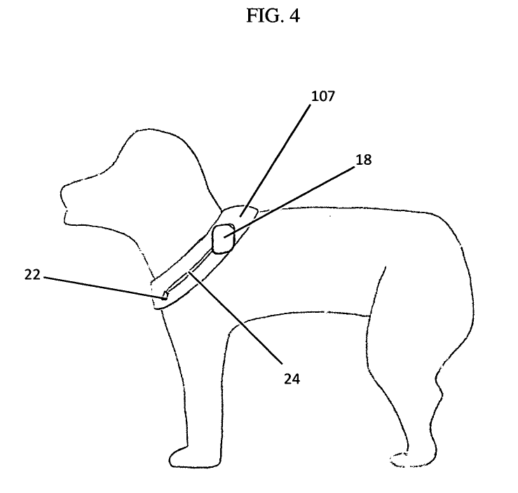

The mounting platform 104 preferably comes in the form of a torso pack 105 as shown in FIG. 3 or a collar 107 as shown in FIG. 4. In the torso pack example of FIG. 3, the outlet 22 is preferably located in a vein or artery along the animal's upper leg (e.g., the cephalic vein). In the collar example in FIG. 4, the outlet 22 is located in a vein or artery along the animal's neck (e.g., the jugular vein). In this way as demonstrated in both specific examples, the outlet 22 is in close proximity with other parts of the mounting platform 104.

The triggering device further includes a first electromagnetic wave transmitter (e.g., a radio transmitter) 112 for transmitting an actuation signal from the actuator 106. Input, output and internal signals of the triggering device are preferably controlled by a first controller 114 (preferably a MIMO controller). The mounting platform 104 includes an electromagnetic wave receiver 116 for receiving the actuation signal(s) from the first transmitter 112. The mounting platform includes a second controller 118 for processing input, output and internal signals, and the second controller 118 relays a received actuation signal to the fluid inducer 20 to cause fluid to flow from the fluid storage chamber 18 to the outlet 22. In this way, the triggering device 102 and mounting platform 104 can communicate wirelessly, allowing an animal to operate untethered to an animal handler.

Preferably, the triggering device 102 includes a light 119 that illuminates when an actuation signal is sent from the triggering device 102 to the mounting platform 104. The triggering device may also include a chronometric device 120 for tracking time (preferably, the times when an actuation signal is sent from the first transmitter 112). The triggering device 102 preferably includes a digital storage medium 122 for storing data associated with the times tracked by the chronometric device 120. The first controller 114 may further include a processor for processing such data. In order to retrieve this data, the triggering device 102 preferably includes a data port 124 (e.g., a USB port or other data transfer port now known or later developed) for downloading data stored on the digital storage medium 122. In one embodiment, the digital storage medium 122 itself is removable (e.g., a USB memory device).

The mounting platform 104 preferably includes a location tracking device 126 (e.g., a GPS device) and a second electromagnetic wave transmitter 128 for transmitting location data corresponding to the tracked location of the mounting platform to a second electromagnetic receiver 130 of the triggering device. The location data can then be stored on the digital storage medium 122 and such data can be associated with the data stored regarding the times when an actuation signal is sent from the triggering device 102. Such data can be downloaded, for example, through the data port 124 described above or via a removable version of the digital storage medium 122.

In addition to the apparatus embodiments described herein, this disclosure also includes a method for rapidly training an animal to act in a specific way. For example, such training may be used to train an animal to identify a specific target odor and to rapidly demonstrate a trained signal after detection of the specific target odor, the method comprising the steps of (1) mounting a mounting platform onto the body of an animal wherein the mounting platform comprises a fluid inducer; a conduit connected to the fluid inducer; and an outlet configured for fluid to flow therethrough to enter the bloodstream of the animal; (2) engaging the outlet in proximity with the bloodstream of the animal; and (3) activating the fluid inducer substantially when the animal displays a desired behavior (e.g., gives a trained signal if the animal is exposed to an odor for which the animal is being trained to give a trained signal). The activating step may further include the step of engaging an actuator of a triggering device to send an actuation signal to an electromagnetic wave receiver of the mounting platform, thereby engaging the fluid inducer to move fluid to the outlet such that the fluid enters the bloodstream of the animal.

The engaging step may further include inserting a removable needle along with an IV catheter into a vein or artery of an animal wherein the IV catheter operates as the outlet 22, and mounting the mounting platform onto the animal (e.g., as a torso pack or a collar).

Additional steps may include tracking the times when an actuation signal is sent to the mounting platform; and storing the tracked times on a digital storage medium. In an embodiment including position tracking technology, additional steps may include tracking the location of the mounting platform; and storing data regarding the location of the mounting platform at least during the tracked times when an actuation signal is sent to the mounting platform.

Advantages of the examples described herein include the ability to rapidly train an animal with positive chemical reinforcement directly to the bloodstream of the animal. Embodiments are available for both leashed and untethered use of the disclosed technology. The described examples allow data to be tracked regarding the number of times actuation signals are sent to train a particular animal, and the duration of the actuation signals can also be tracked to give a sense of the magnitude of each actuation signal. Location data may also be tracked to provide a reference point of where an animal is during a specific time including times when actuation signals are sent to the animal. The data from these actions is stored and can be later reviewed for each individual animal being trained to determine, for example, which animals learn faster with less positive reinforcement feedback.

Different embodiments of the disclosure may include various features as described herein and the specific examples given are not intended to limit the number and type of variations of embodiments contemplated herein.

The foregoing description of preferred embodiments of the present disclosure has been presented for purposes of illustration and description. The described preferred embodiments are not intended to be exhaustive or to limit the scope of the disclosure to the precise form(s) disclosed. Obvious modifications or variations are possible in light of the above teachings. The embodiments are chosen and described in an effort to provide the best illustrations of the principles of the disclosure and its practical application, and to thereby enable one of ordinary skill in the art to utilize the concepts revealed in the disclosure in various embodiments and with various modifications as are suited to the particular use contemplated. All such modifications and variations are within the scope of the disclosure as determined by the appended claims when interpreted in accordance with the breadth to which they are fairly, legally, and equitably entitled.

What is claimed is:

1. An injection apparatus for rapidly training an animal to associate a behavior conducted by the animal with rapid chemical feedback to the animal's bloodstream, the apparatus comprising:
a triggering device to trigger flow of a fluid through the injection apparatus, the triggering apparatus further comprising an actuator; and
a mounting platform for mounting to the body of an animal, the mounting platform further comprising:
a fluid inducer responsive to the actuator;
a fluid storage chamber;
a conduit connected to the fluid inducer and the fluid storage chamber;
an outlet connected to the conduit and configured for fluid to flow therethrough to enter the bloodstream of the animal.

2. The injection apparatus of claim 1 wherein the outlet further comprises an intravenous catheter.

3. The injection apparatus of claim 1 wherein the outlet further comprises a nasal discharge port.

4. The injection apparatus of claim 1 further comprising:
the triggering device further comprising:
a first power source; and
a first electromagnetic wave transmitter powered by the first power source, the first electromagnetic wave transmitter for transmitting an actuation signal;
the mounting platform further comprising:
a second power source; and
a first electromagnetic wave receiver powered by the second power source, the first electromagnetic wave receiver for receiving an actuation signal to activate the fluid inducer.

5. The injection apparatus of claim 4 wherein the triggering device further comprises a light that illuminates when an actuation signal is sent, thereby letting a user know that an actuation signal was transmitted.

6. The injection apparatus of claim 4 wherein the triggering device further comprises a chronometric instrument for tracking time, a digital storage medium for storing data, and a controller in communication with the chronometric instrument and the digital storage medium for recording the timing of the actuation signals as a timing data set onto the digital storage medium.

7. The injection apparatus of claim 6 wherein the triggering device further comprises a second electromagnetic wave receiver and wherein the mounting platform further comprises:
a location tracking device; and
a second electromagnetic wave transmitter for transmitting location data to the triggering device so that location data is sent from the triggering device to the mounting platform and the location data is then associated with the particular times that actuation signals are transmitted, thereby forming a location data set including time and location data when actuation signals are sent from the triggering device.

8. The injection apparatus of claim 6 wherein the triggering device further comprises a port for downloading the timing data set stored on the digital storage medium.

9. The injection apparatus of claim 7 wherein the triggering device further comprises a port for downloading the location data set stored on the digital storage medium.

10. The injection apparatus of claim 6 wherein the digital storage medium of the triggering device further comprises a removable digital storage medium.

11. The injection apparatus of claim 7 wherein the digital storage medium of the triggering device further comprises a removable digital storage medium.

12. A method for rapidly training an animal to associate a behavior conducted by the animal with rapid chemical feedback to the animal's bloodstream, the method comprising the steps of:
mounting a mounting platform onto the body of an animal wherein the mounting platform comprises:
a fluid inducer;
a conduit connected to the fluid inducer; and
an outlet configured for fluid to flow therethrough to enter the bloodstream of the animal;
engaging the outlet in proximity with the bloodstream of the animal; and
activating the fluid inducer substantially when the animal exhibits a specified behavior for which the animal is being trained.

13. The method of claim 12 wherein the activating step further comprises the steps of:
engaging an actuator of a triggering device to send an actuation signal to an electromagnetic wave receiver of the mounting platform, thereby engaging the fluid inducer to move fluid to the outlet such that the fluid enters the bloodstream of the animal.

14. The method of claim 13 further comprising the steps of:
tracking the times when an actuation signal is sent to the mounting platform; and
storing the tracked times on a digital storage medium.

15. The method of claim 14 further comprising the steps of:
tracking the location of the mounting platform; and
storing data regarding the location of the mounting platform at the tracked times when an actuation signal is sent to the mounting platform.
